(19) United States
(12) Patent Application Publication
(10) Pub. No.: US 2007/0190140 A1
(43) Pub. Date: Aug. 16, 2007

(54) Pharmaceutical Composition in the Form of a Gastric-Resident Tablet Containing an Active Principle

(75) Inventors: Gerard Alaux, Beynes (FR); Estelle Chouin, Chilly Mazarin (FR); Nathalie Dufresne-Arokiassamy, Etrechy (FR)

Correspondence Address: ROSS J. OEHLER;SANOFI-AVENTIS U.S. LLC, 1041 ROUTE 202-206, MAIL CODE: D303A, BRIDGEWATER, NJ, 08807, US

(73) Assignee: sanofi-aventis, Paris (FR)

(21) Appl. No.: 11/675,712

(22) Filed: Feb. 16, 2007

Related U.S. Application Data
(63) Continuation of application No. PCT/FR05/02092, filed on Aug. 17, 2005.

(30) Foreign Application Priority Data
Aug. 19, 2004 (FR) 0408986

(51) Int. Cl. A61K 9/22; A61K 31/43; A61K 31/56; A61K 31/165; A61K 31/401; A61K 31/18

(52) U.S. Cl. 424/468; 514/169; 514/192; 514/423; 514/471; 514/617; 514/602

(57) ABSTRACT

The disclosure concerns a pharmaceutical composition in the form of a gastric resident matrix tablet, comprising an active principle, characterized in that when contacted with an environment representing gastric fluid, it increases after 15 minutes in volume, by a swelling of at least 200%.

The subject of the present invention is a pharmaceutical composition in tablet form, containing an active ingredient, which can be used for administration once per day.

Conventional prolonged-release pharmaceutical dosage forms are hardly suitable for certain active ingredients which exhibit an absorption window in the upper parts of the gastrointestinal tract, that is to say which are absorbed in the stomach, the upper parts of the small intestine, duodenum, jejunum and ileum, and less or little in the colon. Indeed, the conventional administrable unit releases the active substance along its passage in the gastrointestinal tract and not only in the part where the absorption of the active ingredient is maximum.

The subject of the present invention is a pharmaceutical composition in the form of a tablet containing an active ingredient, which can be used for administration once per day, overcoming the disadvantages mentioned above.

The invention is characterized in that upon contact with the gastric fluid, the pharmaceutical composition rapidly increases its volume. It is indeed clearly advantageous for this composition to increase its volume not only considerably but also very rapidly as soon as it comes into contact with the gastric fluid. This makes it possible to ensure a longer residence time for this pharmaceutical composition in the stomach, to avoid premature gastric emptying and to ensure that most of the active ingredient contained in the pharmaceutical composition is released and absorbed in the portion of the gastrointestinal tract where the absorption capacity is the greatest.

A subject of the invention consists of a pharmaceutical composition in the form of a matrix tablet, comprising an active ingredient, and allowing prolonged release thereof, which after fifteen minutes of contact with a medium representative of the gastric fluid rapidly increases in volume by a degree of swelling of at least 200%, more particular by at least 250%.

The expression matrix tablet is understood to mean a pharmaceutical composition for oral administration containing an active substance uniformly dispersed in one or more appropriate excipients which, after compression, allow the formation of a matrix capable of controlling the release of the active ingredient.

The expression medium representative of the gastric fluid is understood to mean an aqueous solution containing 0.01 M hydrochloric acid and 0.1 M sodium chloride at 37° C.

The degree of swelling of the tablets is determined by measuring the thickness and the diameter of the dry tablet and of the tablet which has remained for fifteen minutes immersed in the medium representative of the gastric fluid and using a suitable measuring instrument. The degree of swelling (in percent) may thus be expressed in thickness, diameter or volume, according to the following formulae:
Degree of swelling in thickness:
   ((Epₜ₁₅−Epₜ₀)/Epₜ₀)×100
   Epₜ₀=thickness of the tablet at T0
   Epₜ₁₅=thickness of the tablet at 15 minutes
Degree of swelling in diameter:
   ((Dₜ₁₅−Dₜ₀)/Dₜ₀)×100
   Dₜ₀=diameter of the tablet at T0
   Dₜ₁₅=diameter of the tablet at 15 minutes.
Degree of swelling in volume:
   ((Vₜ₁₅−Vₜ₀)/Vₜ₀)×100
   Vₜ₀=volume of the tablet at T0
   Vₜ₁₅=volume of the tablet at 15 minutes.

the volume of the tablet being calculated according to the following formula:
for a convex tablet: 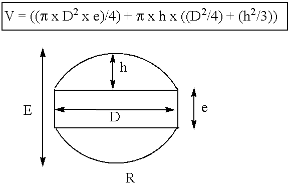
Where D is the diameter of the tablet, e represents the thickness of the slice of the tablet, h represents the half-difference between the total thickness of the tablet and the thickness of the slice and R represents the radius of curvature of the tablet.
for a tablet whose radius of curvature is equal to the diameter (for example, format 10R10 mm, 12R12 mm, and the like):

$$V = ( ( n \times {D}^{2} \times e ) / 4 ) + 0.0359 \times π \times {D}^{3}$$
Where e=E−0.28D.

According to one aspect of the invention, the pharmaceutical composition exists in the form of a single-phase matrix tablet.

According to another aspect of the invention, the pharmaceutical composition exists in the form of a matrix tablet having at least two phases.

According to another aspect of the invention, the pharmaceutical composition may comprise one or more active ingredients in one or more phases. The pharmaceutical composition will comprise more particularly one or two active ingredients.

The expression phase is understood to mean a homogeneous mixture of one or more excipients, in powdered or granule form, which may contain an active ingredient.

A pharmaceutical composition according to the invention comprising two or more phases may exist in the form of a multi-layer (double-layer, triple-layer and the like), more particularly a double-layer, tablet, in the form of a core coated with one or more phases.

According to another subject, the invention consists of a pharmaceutical composition in the form of a gastric retention matrix tablet comprising an active ingredient and at least one phase containing at least, as excipients:
a) povidone and/or polyvinyl acetate in proportions ranging from 30 to 80% by weight of the phase,
b) crospovidone in proportions ranging from 5 to 25% by weight of the phase, and
c) carbomer in proportions ranging from 5 to 40% by weight of the phase.

Alternatively, according to another subject of the invention, the crospovidone may be replaced or combined with another superdisintegrant such as low-substituted hydroxypropyl cellulose (L-HPC), sodium carboxymethyl starch and/or sodium croscarmellose.

The matrix tablet according to the invention has the advantage of swelling very rapidly upon contact with gastric fluids. Indeed, the presence of the excipients a), b) and c) in the proportions according to the invention makes it possible to obtain a swelling synergy. The tablet could thus remain for several hours in the stomach.

When the matrix compound comprises at least two phases, one or more of the phases may comprise an active ingredient. Moreover, each phase may have an identical or different excipient composition from another phase, it being understood that at least one of the phases comprises the excipients a), b) and c) in proportions as indicated according to the invention. When one of the phases does not comprise the excipients a), b) and c), each in the proportions as indicated according to the invention, persons skilled in the art can determine its composition according to the biopharmaceutical needs, such as control of the release of the active ingredient, increase in the degree of swelling.

The povidone and polyvinyl acetate excipients or the povidone/polyvinyl acetate mixture are commercially available or more particularly the mixture is chosen from those marketed under the name Kollidon® SR.

The povidone and/or the polyvinyl acetate are present in a quantity ranging from 30 to 80% by weight of the phase containing it and more particularly from 30 to 65%.

Crospovidone is a crosslinked homopolymer of N-vinyl-2-pyrrolidinone having a molecular weight greater than 1 000 000 DA. This polymer belongs to the category of superdisintegrants capable of rapidly and intensely capturing the surrounding liquid. By way of example, there may be mentioned the crospovidone marketed under the name Kollidon® CL (BASF) or Plasdone® XL (ISP).

Hydroxypropyl cellulose is a low-substituted cellulose hydroxypropyl ether which is insoluble in water and alcohols but which is capable of swelling in these solvents. By way of example, the L-HPC LH-11 grade supplied by Shin Etsu may be mentioned.

Sodium carboxymethyl starch or sodium starch glycolate is the sodium salt of a carboxymethylated ether of starch. It exists in three grades, A, B and C, which differ by their sodium content. By way of example, there may be mentioned the sodium starch glycolate sold under the trade name Primojel® (Avebe) or Explotab® (JRS Pharma).

Sodium croscarmellose is a cellulosic polymer obtained by crosslinking sodium carmellose. By way of example, Ad-Di-Sol® (FMC) may be mentioned.

Crospovidone or the superdisintegrants, such as low-substituted hydroxypropyl cellulose, sodium carboxymethyl starch or sodium starch glycolate, sodium croscarmellose, are present in a quantity ranging from 5 to 25% by weight of the phase containing them and more particularly from 10% to 25%.

The carbomer is a polymer of acrylic acid crosslinked with an allyl ether of sucrose or of pentaerythritol having a very high molecular weight (of the order of a million). By way of example, there may be mentioned Carbopol® 974 or Carbopol® 71G (NOVEON), more particularly Carbopol® 71G which makes it possible to obtain aqueous dispersions having a viscosity of between 4000 and 11 000 csp (dispersion at 0.5%). The carbomer is present in the tablet or in a phase in proportions in a quantity ranging from 5 to 40% by weight respectively of the tablet or of the phase and more particularly from 10 to 35%.

According to another subject of the invention, the excipients a), b) and c) are present respectively in quantities of 40 to 70% for povidone and/or polyvinyl acetate, 10 to 20% for crospovidone and 10 to 30% for the carbomer.

The tablet may also comprise any excipient which is suitable and necessary for the manufacture of the tablet, such as:
soluble or insoluble diluents (microcrystalline cellulose, lactose, mannitol, dicalcium phosphate and the like), more particularly insoluble diluents such as microcrystalline cellulose, in a quantity ranging from 5 to 30% by weight of the phase containing it;
lubricants (magnesium stearate, talc, hydrogenated castor oil, PEG 6000, glyceryl behenate, stearic acid and the like), and
glidants (colloidal silica, precipitated silica and the like).

The pharmaceutical compositions according to the invention may for example be useful for benzamides and α1-antagonists, and the following active ingredients: captopril, furosemide, ursodesoxycholic acid, amoxicillin, (+)-α-aminomethyl-2-methoxysulphonamidobenzenemethanol (disclosed in patent application EP 842 148 in Example 3.6) or 3′-(2-amino-1-hydroxy-ethyl)-4′-fluoromethanesulphonanilide (NS 49).

The benzamides are in particular metoclopramide, veralipride, alizapride, clebopride and more particularly amisulpride, tiapride, sulpiride and their salts.

The α1-antagonists are in particular terazosin and alfuzosin and their salts, in particular alfuzosin hydrochloride. They are intended in particular for the treatment of benign prostatic hypertrophy.

Captopril is used in particular for the treatment of hypertension, furosemide as a diuretic, amoxicillin and its salts as an antibiotic, and ursodesoxycholic acid and its salts is used for the treatment of cholelithiases, hepatic disorders and syphilis.

For the purposes of the present invention, the various enantiomers or diastereoisomers of the various active ingredients or families of active ingredients (benzamides, α1-antagonists) are also covered, including mixtures thereof, in particular their racemic mixtures, but also their salts.

Among the active ingredients which are more particularly suitable for the compositions according to the invention, there may be mentioned amisulpride (D)-tartrate, (S)-(−)-amisulpride, (S)-(−)-amisulpride (D)-tartrate, tiapride hydrochloride, alfuzosin hydrochloride and 3′-(2-amino-1-hydroxyethyl)-4′-fluoromethanesulphonanilide hydrochloride.

The quantity of active ingredient transported in the pharmaceutical composition is in general from 0.1 mg to 200 mg.

The tablets of the invention may be produced by direct compression of a mixture of the powders or by granulation followed by compression using the customary production technologies. The compression format chosen may be optimized according to the general knowledge of persons skilled in the art.

The working compression force varies between 500 DaN and 3000 DaN so as to obtain tablets having a breaking strength which makes it possible to handle them and to administer them without any problem (between 80 and 300 N for 10R10 mm tablets for example). Tablets with a single phase or with at least two phases having a shape which allows easy administration and swallowing are obtained according to methods which will be described in greater detail in the examples.

Depending on the quantity of active substance which is transported, each phase of the tablet may have a different thickness ranging from 1 to 8 mm, but preferably from 2 mm to 6 mm. These sizes may vary according to the compression format.

A coating made of polymeric materials may be additionally applied to the pharmaceutical preparation which have the aim of simply protecting or varying the kinetics of release of the active ingredient from the polymeric matrix, according to techniques well known to persons skilled in the art.

The examples which follow are intended to illustrate the invention.

### EXAMPLE 1

### Preparation of a Single-layer Tablet Comprising 10 mg of Alfuzosin Hydrochloride

| | | | Unit |
|---|---|---|---|
| Excipient | | | composition |
| Pharmacopoeia | Excipient | Percentage | (300 mg |
| name | trade name | composition | tablets) |
| Alfuzosin | Alfuzosin | 3.33% | 10.00 mg |
| hydrochloride | hydrochloride | | |
| Mixture | Kollidon ® SR | 60.00% | 180.00 mg |
| povidone (19%) | | | |
| and polyvinyl | | | |
| acetate (80%) | | | |
| Crospovidone | Kollidon ® CL | 15.00% | 45.00 mg |
| Carbomer | Carbopol ® 71G | 20.47% | 61.40 mg |
| Colloidal | Aerosil ® | 0.20% | 0.60 mg |
| silica | | | |
| Magnesium | | 1.00% | 3.00 mg |
| stearate | | | |

Method of Manufacture

The tablets described in this example are obtained by direct compression using a Forgerais OA alternating tableting machine.

All the excipients are sieved (1 mm mesh) beforehand and then mixed using a Turbula® mixer. The mixture flows freely and facilitates the filling of the compression chamber.

Mixture and Tablet Characteristics
Flow of the mixture: <10 seconds per 100 g of mixture.
Hardness of the tablets: 100 Newtons.
Mass of the tablets: 300 mg
Format of the tablets: 10R10 mm.

The degree of swelling of the tablets is determined by measuring the thickness and the diameter of the dry tablet and of the tablet which has been left for fifteen minutes immersed in gastric fluid at 37° C. (0.01 M HCl +0.1 M NaCl), using a suitable measuring instrument. In the example described, the degree of swelling is 80% by thickness and 25% by diameter, that is about 200% by volume.

The profile of release of an active ingredient into the gastric medium (pH 2+0.1 M NaCl) obtained with this formulation is a profile of the order 0, that is:
10 to 20% released in 1 hour.
40 to 55% released in 6 hours.
65 to 85% in 12 hours.
85 to 100% released in 20 hours.

### Example 2

### Preparation of a Double-layer Tablet Comprising 10 mg of Alfuzosin Hydrochloride

| Excipient | | | |
|---|---|---|---|
| Pharmacopoeia | Excipient | Percentage | Unit composition |
| name | trade name | composition | (500 mg tablets) |
| Layer 1 | | | 300 mg |
| Alfuzosin | Alfuzosin | 3.33% | 10.00 mg |
| hydrochloride | hydrochloride | | |
| Mixture povidone | Kollidon ® SR | 50.00% | 150.00 mg |
| and polyvinyl | | | |
| acetate | | | |
| Microcrystalline | Avicel ® | 10.00% | 30.00 mg |
| cellulose | PH 102 | | |
| Crospovidone | Kollidon ® CL | 15.00% | 45.00 mg |
| Carbomer | Carbopol ® 71G | 20.47% | 61.40 mg |
| Colloidal silica | Aerosil 200 ® | 0.20% | 0.60 mg |
| Magnesium | Magnesium | 1.00% | 3.00 mg |
| stearate | stearate | | |
| Layer 2 | | | 200.00 mg |
| Mixture povidone | Kollidon ® SR | 63.33% | 126.70 mg |
| and polyvinyl | | | |
| acetate | | | |
| Crospovidone | Kollidon ® CL | 15.00% | 30.00 mg |
| Carbomer | Carbopol ® 71G | 20.47% | 40.90 mg |
| Colloidal silica | Aerosil 200 ® | 0.20% | 0.40 mg |
| Magnesium | Magnesium | 1.00% | 2.00 mg |
| stearate | stearate | | |

Method of Manufacture

The tablets described in this example are obtained by direct compression using a Forgerais OA alternating tableting machine.

All the excipients are sieved (1 mm mesh) beforehand and then mixed using a Turbula® mixer. The mixture flows freely and facilitates the filling of the compression chamber.

Mixture and Tablet Characteristics
Flow of the mixture: <10 seconds per 100 g of mixture.
Hardness of the tablets: 125 Newtons.
Mass of the tablets: 500 mg
Format of the tablets: 12R12 mm.

The degree of swelling of the tablets is determined by the method described above. In this example, the swelling is 80% by thickness and 30% by diameter, that is about 300% by volume.

The profile of release of an active ingredient into the gastric medium (pH 2+0.1 M NaCl) obtained with this formulation is a profile of the order 0, that is:
10 to 20% released in 1 hour.
40 to 55% released in 6 hours.
65 to 85% in 12 hours.
85 to 100% released in 20 hours.

### EXAMPLE 3

### Preparation of a Double-layer Tablet Comprising 10 mg of Alfuzosin Hydrochloride

| Excipient | | | |
|---|---|---|---|
| Pharmacopoeia | Excipient | Percentage | Unit composition |
| name | trade name | composition | (500 mg tablets) |
| Phase 1 | | | 200.00 mg |
| Alfuzosin | Alfuzosin | 5.00% | 10.00 mg |
| hydrochloride | hydrochloride | | |
| Mixture povidone | Kollidon ® SR | 34.25% | 68.50 mg |
| and polyvinyl | | | |
| acetate | | | |
| Crospovidone | Kollidon ® CL | 19.25% | 38.50 mg |
| Carbomer | Carbopol ® 71G | 29.25% | 58.50 mg |
| Microcrystalline | Avicel ® | 10.75% | 21.50 mg |
| cellulose | PH 102 | | |
| Colloidal silica | Aerosil 200 ® | 0.30% | 0.60 mg |
| Yellow iron | Yellow iron | 0.20% | 0.40 mg |
| oxide | oxide | | |
| Mg stearate | Mg stearate | 1.00% | 2.00 mg |
| Layer 2 | | | 300.00 mg |
| Mixture povidone | Kollidon ® SR | 35.50% | 106.50 mg |
| and polyvinyl | | | |
| acetate | | | |
| Crospovidone | Kollidon ® CL | 20.50% | 61.50 mg |
| Carbomer | Carbopol ® 71G | 30.50% | 91.50 mg |
| Microcrystalline | Avicel ® | 12.00% | 36.00 mg |
| cellulose | PH 102 | | |
| Colloidal silica | Aerosil 200 ® | 0.30% | 0.90 mg |
| Red iron oxide | Red iron | 0.20% | 0.60 mg |
| | oxide | | |
| Mg stearate | Mg stearate | 1.00% | 3.00 mg |

Method of Manufacture

The tablets described in this example are obtained by direct compression using a Forgerais OA alternating tableting machine.

All the excipients are sieved (1 mm mesh) beforehand and then mixed using a Turbula® inversion mixer. The mixture flows freely and facilitates the filling of the compression chamber.

Mixture and Tablet Characteristics
Flow of the mixture: <10 seconds per 100 g of mixture.
Hardness of the tablets: 130 Newtons.
Mass of the tablets: 500 mg
Format of the tablets: 12R12 mm.

The degree of swelling of the tablets is determined by the method described above. In this example, the swelling is 300% by volume.

### EXAMPLE 4

### Preparation of a 500 mg Double-layer Tablet Comprising 10 mg of Alfuzosin Hydrochloride

| | | | Unit |
|---|---|---|---|
| Excipient | | | composition |
| Pharmacopoeia | Excipient | | (500 mg |
| name | trade name | Percentage | tablets) |
| Layer 1 | | | 200.00 mg |
| Alfuzosin | Alfuzosin | 5.00% | 10.00 mg |
| hydrochloride | hydrochloride | | |
| Mixture povidone | Kollidon ® SR | 33.91% | 67.82 mg |
| and polyvinyl | | | |
| acetate | | | |
| Crospovidone | Kollidon ® CL | 11.14% | 22.28 mg |
| Carbomer | Carbopol ® 71G | 29.07% | 58.14 mg |
| Microcrystalline | Avicel ® | 19.38% | 38.76 mg |
| cellulose | PH 102 | | |
| Colloidal silica | Aerosil 200 ® | 0.30% | 0.60 mg |
| Yellow iron | Yellow iron | 0.20% | 0.40 mg |
| oxide | oxide | | |
| Magnesium | Magnesium | 1.00% | 2.00 mg |
| stearate | stearate | | |
| Layer 2 | | | 300.00 mg |
| Mixture povidone | Kollidon ® SR | 35.73% | 107.19 mg |
| and polyvinyl | | | |
| acetate | | | |
| Crospovidone | Kollidon ® CL | 11.74% | 35.22 mg |
| Carbomer | Carbopol ® 71G | 30.62% | 91.86 mg |
| Microcrystalline | Avicel ® | 20.41% | 61.23 mg |
| cellulose | PH 102 | | |
| Colloidal silica | Aerosil 200 ® | 0.30% | 0.90 mg |
| Red iron oxide | Red iron | 0.20% | 0.60 mg |
| | oxide | | |
| Magnesium | Magnesium | 1.00% | 3.00 mg |
| stearate | stearate | | |

Method of Manufacture

The tablets described in this example are obtained by direct compression using a Forgerais OA alternating tableting machine.

All the excipients are sieved (1 mm mesh) beforehand and then mixed using a Turbula® inversion mixer. The mixture flows freely and facilitates the filling of the compression chamber.

Mixture and Tablet Characteristics
Flow of the mixture: <10 seconds per 100 g of mixture.
Hardness of the tablets: 150 Newtons.
Mass of the tablets: 500 mg
Format of the tablets: 12R12 mm.

The degree of swelling of the tablets is determined by the method described above in Example 1. In this example, the swelling is 350% by volume.

### EXAMPLE 5

### Preparation of a 500 mg Double-layer Tablet Comprising 10 mg of Alfuzosin Hydrochloride

| Excipient | | | |
|---|---|---|---|
| Pharmacopoeia | Excipient | Percentage | Unit composition |
| name | trade name | composition | (500 mg tablets) |
| Layer 1 | | | 200.00 mg |
| Alfuzosin | Alfuzosin | 5.00% | 10.00 mg |
| hydrochloride | hydrochloride | | |
| Mixture povidone | Kollidon ® SR | 33.91% | 67.82 mg |
| and polyvinyl | | | |
| acetate | | | |
| Crospovidone | Kollidon ® CL | 19.38% | 38.76 mg |
| Carbomer | Carbopol ® 71G | 20.83% | 41.66 mg |
| Microcrystalline | Avicel ® | 19.38% | 38.76 mg |
| cellulose | PH 102 | | |
| Colloidal silica | Aerosil 200 ® | 0.30% | 0.60 mg |
| Yellow iron | Yellow iron | 0.20% | 0.40 mg |
| oxide | oxide | | |
| Mg stearate | Mg stearate | 1.00% | 2.00 mg |
| Layer 2 | | | 300.00 mg |
| Mixture povidone | Kollidon ® SR | 35.73% | 107.19 mg |
| and polyvinyl | | | |
| acetate | | | |
| Crospovidone | Kollidon ® CL | 20.41% | 61.23 mg |
| Carbomer | Carbopol ® 71G | 21.95% | 65.85 mg |
| Microcrystalline | Avicel ® | 20.41% | 61.23 mg |
| cellulose | PH 102 | | |
| Colloidal silica | Aerosil 200 ® | 0.30% | 0.90 mg |
| Red iron oxide | Red iron | 0.20% | 0.60 mg |
| | oxide | | |
| Mg stearate | Mg stearate | 1.00% | 3.00 mg |

Method of Manufacture

The tablets described in this example are obtained by direct compression using a Forgerais OA alternating tableting machine.

All the excipients are sieved (1 mm mesh) beforehand and then mixed using a Turbula® inversion mixer. The mixture flows freely and facilitates the filling of the compression chamber.

Mixture and Tablet Characteristics
Flow of the mixture: <10 seconds per 100 g of mixture.
Hardness of the tablets: 150 Newtons.
Mass of the tablets: 500 mg
Format of the tablets: 12R12 mm.

The degree of swelling of the tablets is determined by the method described above. In this example, the swelling is 360% by volume.

### EXAMPLE 6

### Preparation of a 500 mg Double-layer Tablet Comprising 10 mg of Alfuzosin Hydrochloride

| Excipient | | | |
|---|---|---|---|
| Pharmacopoeia | Excipient | Percentage | Unit composition |
| name | trade name | composition | (500 mg tablets) |
| Layer 1 | | | 200.00 mg |
| Alfuzosin | Alfuzosin | 5.00% | 10.00 mg |
| hydrochloride | hydrochloride | | |
| Mixture povidone | Kollidon ® SR | 49.74% | 99.48 mg |
| and polyvinyl | | | |
| acetate | | | |
| Crospovidone | Kollidon ® CL | 19.37% | 38.74 mg |
| Carbomer | Carbopol ® 71G | 15.83% | 31.66 mg |
| Microcrystalline | Avicel ® | 8.56% | 17.12 mg |
| cellulose | PH 102 | | |
| Colloidal silica | Aerosil 200 ® | 0.30% | 0.60 mg |
| Yellow iron | Yellow iron | 0.20% | 0.40 mg |
| oxide | oxide | | |
| Magnesium | Magnesium | 1.00% | 2.00 mg |
| stearate | stearate | | |
| Layer 2 | | | 300.00 mg |
| Mixture povidone | Kollidon ® SR | 52.40% | 157.20 mg |
| and polyvinyl | | | |
| acetate | | | |
| Crospovidone | Kollidon ® CL | 20.41% | 61.23 mg |
| Carbomer | Carbopol ® 71G | 16.68% | 50.04 mg |
| Microcrystalline | Avicel ® | 9.01% | 27.03 mg |
| cellulose | PH 102 | | |
| Colloidal silica | Aerosil 200 ® | 0.30% | 0.90 mg |
| Red iron oxide | Red iron | 0.20% | 0.60 mg |
| | oxide | | |
| Magnesium | Magnesium | 1.00% | 3.00 mg |
| stearate | stearate | | |

Method of Manufacture

The tablets described in this example are obtained by direct compression using a Forgerais OA alternating tableting machine.

All the excipients are sieved (1 mm mesh) beforehand and then mixed using a Turbula® inversion mixer. The mixture flows freely and facilitates the filling of the compression chamber.

Mixture and Tablet Characteristics
Flow of the mixture: <10 seconds per 100 g of mixture.
Hardness of the tablets: 140 Newtons.
Mass of the tablets: 500 mg
Format of the tablets: 12R12 mm.

The degree of swelling of the tablets is determined by the method described above. In this example, the swelling is 270% by volume.

### EXAMPLE 7

### Preparation of a Double-layer Tablet Comprising 10 mg of Alfuzosin Hydrochloride

| Excipient | | | |
|---|---|---|---|
| Pharmacopoeia | Excipient | Percentage | Unit composition |
| name | trade name | composition | (500 mg tablets) |
| Layer 1 | | | 200.00 mg |
| Alfuzosin | Alfuzosin | 5.00% | 10.00 mg |
| hydrochloride | hydrochloride | | |
| Mixture povidone | Kollidon ® SR | 44.47% | 88.94 mg |
| and polyvinyl | | | |
| acetate | | | |
| Crospovidone | Kollidon ® CL | 11.92% | 23.84 mg |
| Carbomer | Carbopol ® 71G | 17.73% | 35.46 mg |
| Microcrystalline | Avicel ® | 19.38% | 38.76 mg |
| cellulose | PH 102 | | |
| Colloidal silica | Aerosil 200 ® | 0.30% | 0.60 mg |
| Yellow iron | Yellow iron | 0.20% | 0.40 mg |
| oxide | oxide | | |
| Magnesium | Magnesium | 1.00% | 2.00 mg |
| stearate | stearate | | |
| Layer 2 | | | 300.00 mg |
| Mixture povidone | Kollidon ® SR | 46.85% | 140.55 mg |
| and polyvinyl | | | |
| acetate | | | |
| Crospovidone | Kollidon ® CL | 12.56% | 37.68 mg |
| Carbomer | Carbopol ® | 18.68% | 56.04 mg |
| | 71G | | |
| Microcrystalline | Avicel ® | 20.41% | 61.23 mg |
| cellulose | PH 102 | | |
| Colloidal silica | Aerosil 200 ® | 0.30% | 0.90 mg |
| Red iron oxide | Red iron | 0.20% | 0.60 mg |
| | oxide | | |
| Magnesium | Magnesium | 1.00% | 3.00 mg |
| stearate | stearate | | |

Method of Manufacture

The tablets described in this example are obtained by direct compression using a Forgerais OA alternating tableting machine. All the excipients are sieved (1 mm mesh) beforehand and then mixed using a Turbula® inversion mixer. The mixture flows freely and facilitates the filling of the compression chamber.

Mixture and Tablet Characteristics
Flow of the mixture: <10 seconds per 100 g of mixture.
Hardness of the tablets: 150 Newtons.
Mass of the tablets: 500 mg
Format of the tablets: 12R12 mm.

The degree of swelling of the tablets is determined by the method described above. In this example, the swelling is 300% by volume.

### EXAMPLE 8

### Preparation of a 500 mg Double-layer Tablet Comprising 10 mg of Alfuzosin Hydrochloride

| Excipient | | | |
|---|---|---|---|
| Pharmacopoeia | Excipient | Percentage | Unit composition |
| name | trade name | composition | (500 mg tablets) |
| Layer 1 | | | 200.00 mg |
| Alfuzosin | Alfuzosin | 5.00% | 10.00 mg |
| hydrochloride | hydrochloride | | |
| Mixture povidone | Kollidon ® SR | 49.74% | 99.48 mg |
| and polyvinyl | | | |
| acetate | | | |
| Crospovidone | Kollidon ® CL | 19.37% | 38.74 mg |
| Carbomer | Carbopol ® 71G | 15.83% | 31.66 mg |
| Microcrystalline | Avicel ® | 8.56% | 17.12 mg |
| cellulose | PH 102 | | |
| Colloidal silica | Aerosil 200 ® | 0.30% | 0.60 mg |
| Yellow iron | Yellow iron | 0.20% | 0.40 mg |
| oxide | oxide | | |
| Magnesium | Magnesium | 1.00% | 2.00 mg |
| stearate | stearate | | |
| Layer 2 | | | 300.00 mg |
| Mixture povidone | Kollidon ® SR | 52.40% | 157.20 mg |
| and polyvinyl | | | |
| acetate | | | |
| Crospovidone | Kollidon ® CL | 20.41% | 61.23 mg |
| Carbomer | Carbopol ® | 16.68% | 50.04 mg |
| | 71G | | |
| Microcrystalline | Avicel ® | 9.01% | 27.03 mg |
| cellulose | PH 102 | | |
| Colloidal silica | Aerosil 200 ® | 0.30% | 0.90 mg |
| Red iron oxide | Red iron | 0.20% | 0.60 mg |
| | oxide | | |
| Magnesium | Magnesium | 1.00% | 3.00 mg |
| stearate | stearate | | |

Method of Manufacture

The tablets described in this example are obtained by direct compression using a Forgerais OA alternating tableting machine.

All the excipients are sieved (1 mm mesh) beforehand and then mixed using a Turbula® inversion mixer. The mixture flows freely and facilitates the filling of the compression chamber.

Mixture and Tablet Characteristics
Flow of the mixture: <10 seconds per 100 g of mixture.
Hardness of the tablets: 150 Newtons.
Mass of the tablets: 500 mg
Format of the tablets: 12R12 mm.

The degree of swelling of the tablets is determined by the method described above. In this example, the swelling is 300% by volume.

The following Examples 9 and 10 present placebo single-layer tablet compositions. These compositions may be used as a swelling placebo layer in the context of the manufacture of multi-layer tablets. It is also possible to incorporate the active ingredient into these compositions, for example, in an amount of 10 mg so as to obtain a pharmaceutical composition according to the invention.

### EXAMPLE 9

### Preparation of a 500 mg Single-layer Placebo Tablet

| Excipient | | | |
|---|---|---|---|
| Pharmacopoeia | Excipient | Percentage | Unit composition |
| name | trade name | composition | (500 mg tablets) |
| Mixture povidone | Kollidon ® SR | 35.73% | 178.65 mg |
| and polyvinyl | | | |
| acetate | | | |
| Sodium starch | Primojel ® | 20.41% | 102.05 mg |
| glycolate | | | |
| Carbomer | Carbopol ® 71G | 21.95% | 109.75 mg |
| Microcrystalline | Avicel ® | 20.41% | 102.05 mg |
| cellulose | PH 102 | | |
| Colloidal silica | Aerosil 200 ® | 0.30% | 1.50 mg |
| Red iron oxide | Red iron | 0.20% | 1.00 mg |
| | oxide | | |
| Magnesium | Magnesium | 1.00% | 5.00 mg |
| stearate | stearate | | |

| | | |
|---|---|---|
| Total | 100.00% | 500.00 mg |

Method of Manufacture

The tablets described in this example are obtained by direct compression using a Forgerais OA alternating tableting machine.

All the excipients are sieved (1 mm mesh) beforehand and then mixed using a Turbula® inversion mixer. The mixture flows freely and facilitates the filling of the compression chamber.

Mixture and Tablet Characteristics
Flow of the mixture: <10 seconds per 100 g of mixture.
Hardness of the tablets: 140 Newtons.
Mass of the tablets: 500 mg
Format of the tablets: 12R12 mm.

The degree of swelling of the tablets is determined by the method described above. In this example, the swelling is 340% by volume.

### EXAMPLE 10

### Preparation of a 500 mg Single-layer Placebo Tablet

| Excipient | | | |
|---|---|---|---|
| Pharmacopoeia | Excipient | Percentage | Unit composition |
| name | trade name | composition | (500 mg tablets) |
| Mixture povidone | Kollidon ® SR | 35.73% | 178.65 mg |
| and polyvinyl | | | |
| acetate | | | |
| L-HPC | LH-11 | 20.41% | 102.05 mg |
| Carbomer | Carbopol ® 71G | 21.95% | 109.75 mg |
| Microcrystalline | Avicel ® | 20.41% | 102.05 mg |
| cellulose | PH 102 | | |
| Colloidal silica | Aerosil 200 ® | 0.30% | 1.50 mg |
| Red iron oxide | Red iron | 0.20% | 1.00 mg |
| | oxide | | |
| Magnesium | Magnesium | 1.00% | 5.00 mg |
| stearate | stearate | | |

| | | |
|---|---|---|
| Total | 100.00% | 500.00 mg |

Method of Manufacture

The tablets described in this example are obtained by direct compression using a Forgerais OA alternating tableting machine.

All the excipients are sieved (1 mm mesh) beforehand and then mixed using a Turbula® inversion mixer. The mixture flows freely and facilitates the filling of the compression chamber.

Mixture and Tablet Characteristics
Flow of the mixture: <10 seconds per 100 g of mixture.
Hardness of the tablets: 140 Newtons.
Mass of the tablets: 500 mg
Format of the tablets: 12R12 mm.

The degree of swelling of the tablets is determined by the method described above. In this example, the swelling is 470% by volume.

### EXAMPLE 11

### Preparation of a 700 mg Triple-layer Tablet Containing 10 mg of Alfuzosin Hydrochloride

| | Percentage composition % | Formula |
|---|---|---|
| Layer 1 placebo | | |
| Kollidon ® SR | 35.73 | 107.19 |
| Kollidon ® CL | 20.41 | 61.23 |
| Carbopol ® 71 G | 21.95 | 65.85 |
| Red iron oxide | 0.20 | 0.60 |
| Avicel ® pH 102 | 20.41 | 61.23 |
| Aerosil 200 ® | 0.30 | 0.90 |
| Magnesium stearate | 1.00 | 3.00 |
| Total | 100.00 | 300.00 |
| Active layer | | |
| Alfuzosin HCl | 5.00 | 10.00 |
| Kollidon ® SR | 33.91 | 67.82 |
| Kollidon ® CL | 19.38 | 38.76 |
| Carbopol ® 71 G | 20.83 | 41.66 |
| Avicel ® pH 102 | 19.38 | 38.76 |
| Yellow iron oxide | 0.20 | 0.40 |
| Aerosil 200 ® | 0.30 | 0.60 |
| Mg stearate | 1.00 | 2.00 |
| Total | 100.00 | 200.00 |
| Placebo layer | | |
| Kollidon ® SR | 35.73 | 71.46 |
| Kollidon ® CL | 20.41 | 40.82 |
| Carbopol ® 71 G | 21.95 | 43.90 |
| Red iron oxide | 0.20 | 0.40 |
| Avicel ® pH 102 | 20.41 | 40.82 |
| Aerosil 200 ® | 0.30 | 0.60 |
| Magnesium stearate | 1.00 | 2.00 |
| Total | 100.00 | 200.00 |

Method of Manufacture

The tablets described in this example are obtained by direct compression using a Forgerais OA alternating tableting machine.

All the excipients are sieved (1 mm mesh) beforehand and then mixed using a Turbula® inversion mixer. The mixture flows freely and facilitates the filling of the compression chamber.

Mixture and Tablet Characteristics
Flow of the mixture: <10 seconds per 100 g of mixture.
Hardness of the tablets: 200 Newtons.
Mass of the tablets: 700 mg
Format of the tablets: 18*9R7 mm.

The degree of swelling of the tablets is determined by the method described above. In this example, the swelling is 104% by thickness, 41% by width and 36% by length.

What is claimed is:

1. A pharmaceutical composition in the form of a gastric retention matrix tablet comprising an active ingredient, where upon contact with a medium representative of the gastric fluid, said tablet increases in volume, after fifteen minutes, by a degree of swelling of at least 200%.

2. A pharmaceutical composition in the form of a gastric retention matrix tablet according to claim 1, comprising one or more phases.

3. A pharmaceutical composition in the form of a gastric retention matrix tablet according to claim 2, wherein at least one of the phases contains at least, as excipients:
a) povidone and/or polyvinyl acetate in proportions ranging from 30 to 80% by weight of the phase,
b) crospovidone in proportions ranging from 5 to 25% by weight of the phase, and
c) carbomer in proportions ranging from 5 to 40% by weight of the phase.

4. A pharmaceutical composition in the form of a matrix tablet according to claim 3, wherein the povidone and/or polyvinyl acetate is present in proportions ranging from 30 to 65% by weight of the pharmaceutical composition.

5. A pharmaceutical composition in the form of a matrix tablet comprising an active ingredient, according to claim 3, wherein the crospovidone is present in proportions ranging from 10 to 25% by weight of the pharmaceutical composition.

6. A pharmaceutical composition in the form of a matrix tablet comprising an active ingredient, according to claim 4, wherein the crospovidone is present in proportions ranging from 10 to 25% by weight of the pharmaceutical composition.

7. A pharmaceutical composition in the form of a matrix tablet according to claim 3, wherein the carbomer is present in proportions ranging from 10 to 35% by weight of the pharmaceutical composition.

8. A pharmaceutical composition in the form of a matrix tablet according to claim 4, wherein the carbomer is present in proportions ranging from 10 to 35% by weight of the pharmaceutical composition.

9. A pharmaceutical composition in the form of a matrix tablet according to claim 5, wherein the carbomer is present in proportions ranging from 10 to 35% by weight of the pharmaceutical composition.

10. A pharmaceutical composition in the form of a gastric retention matrix tablet having one or more phases comprising an active ingredient, where upon contact with a medium representative of the gastric fluid, said tablet increases in volume, after fifteen minutes, by a degree of swelling of at least 200%, wherein at least one of the phases contains at least, as excipients:
a) povidone and/or polyvinyl acetate in proportions ranging from 30 to 80% by weight of the phase,
b) crospovidone or another superdisintegrant chosen from low-substituted hydroxypropyl cellulose (L-HPC), sodium carboxymethyl starch and/or sodium croscarmellose or a combination of said crospovidone and said superdisintegrant in proportions ranging from 5 to 25% by weight of the phase, and
c) carbomer in proportions ranging from 5 to 40% by weight of the phase.

11. A pharmaceutical composition in the form of a matrix tablet comprising an active ingredient, according to claim 10, wherein the crospovidone or the superdisintegrant or the combination of the crospovidone and the superdisintegrant is present in proportions ranging from 10 to 25% by weight of the pharmaceutical composition.

12. A pharmaceutical composition in the form of a matrix tablet according to claim 1, containing a diluent in a quantity of 5 to 30%.

13. A pharmaceutical composition in the form of a matrix tablet according to claim 3, further containing a diluent in a quantity of 5 to 30%.

14. A pharmaceutical composition in the form of a matrix tablet according to claim 11, further containing a diluent in a quantity of 5 to 30%.

15. A pharmaceutical composition in the form of a matrix tablet according to claim 1, wherein the active ingredient is chosen from the group consisting of benzamides, α1-antagonists, captopril, furosemide, ursodesoxycholic acid, amoxicillin, (+)-α-aminomethyl-2-methoxysulphonamidobenzenemethanol and 3′-(2-amino-1-hydroxyethyl)-4′-fluoromethanesulphonanilide.

16. A pharmaceutical composition in the form of a matrix tablet according to claim 3, wherein the active ingredient is chosen from the group consisting of benzamides, α1-antagonists, captopril, furosemide, ursodesoxycholic acid, amoxicillin, (+)-α-aminomethyl-2-methoxysulphonamidobenzenemethanol and 3′-(2-amino-1-hydroxyethyl)-4′-fluoromethanesulphonanilide.

17. A pharmaceutical composition in the form of a matrix tablet according to claim 7, wherein the active ingredient is chosen from the group consisting of benzamides, α1-antagonists, captopril, furosemide, ursodesoxycholic acid, amoxicillin, (+)-α-aminomethyl-2-methoxysulphonamidobenzenemethanol and 3′-(2-amino-1-hydroxyethyl)-4′-fluoromethanesulphonanilide.

18. A pharmaceutical composition in the form of a matrix tablet according to claim 15, wherein the active ingredient is alfuzosin hydrochloride.

19. A pharmaceutical composition in the form of a matrix tablet according to claim 16, wherein the active ingredient is alfuzosin hydrochloride.

20. A pharmaceutical composition in the form of a matrix tablet according to claim 15, wherein the active ingredient is present in a quantity ranging from 0.1 mg to 200 mg.

21. A pharmaceutical composition in the form of a matrix tablet according to claim 19, wherein the active ingredient is present in a quantity ranging from 0.1 mg to 200 mg.

22. A pharmaceutical composition in the form of a matrix tablet according to claim 1, wherein the matrix tablet is present in the form of a double-layer matrix tablet comprising two phases.

23. A pharmaceutical composition in the form of a matrix tablet according to claim 3, wherein the matrix tablet is present in the form of a double-layer matrix tablet comprising two phases.

24. A pharmaceutical composition in the form of a matrix tablet according to claim 1, comprising 3.33% alfuzosin hydrochloride, 60.00% of a mixture of 19% povidone and 80% polyvinyl acetate, 15.00% crospovidone, 20.47% carbomer, 0.20% colloidal silica, and 1.00% magnesium stearate.

25. A pharmaceutical composition in the form of a matrix tablet according to claim 23, wherein one phase comprises 3.33% alfuzosin hydrochloride, 50.00% of a mixture of povidone and polyvinyl acetate, 10.00% microcrystalline cellulose, 15.00% crospovidone, 20.47% carbomer, 0.20% colloidal silica, and 1.00% magnesium stearate; and a second phase comprises 63.3% of a mixture of povidone and polyvinyl acetate, 15.00% crospovidone, 20.47% carbomer, 0.20% colloidal silica, and 1.00% magnesium stearate.

26. A pharmaceutical composition in the form of a matrix tablet according to claim 1, comprising:
a placebo layer comprising 35.73% of a mixture povidone and polyvinyl acetate, 20.41%, crospovidone, 21.95% carbomer, 0.20% red iron oxide, 20.41% microcrystalline cellulose, 0.30% colloidal silica, and 1.00% magnesium stearate; and
an active layer comprising 5.00% alfuzosin HCl, 33.91% of a mixture of povidone and polyvinyl acetate, 19.38% crospovidone, 20.83% carbomer, 19.38% microcrystalline cellulose, 0.20% yellow iron oxide, 0.30% colloidal silica, and 1.00% magnesium stearate; and
a second placebo layer comprising 35.73% of a mixture of povidone and polyvinyl acetate, 20.41% crospovidone, 21.95% carbomer, 0.20% microcrystalline cellulose, 20.41% red iron oxide, 0.30% colloidal silica, and 1.00% magnesium stearate.
